(19) United States
(12) Patent Application Publication
(10) Pub. No.: US 2008/0031828 A1
(43) Pub. Date: Feb. 7, 2008

(54) Method for the Treatment of Dyspnea Comprising Combined Administration of Tiotropium Salts and Salts of Salmeterol

(75) Inventor: Piet Cornelissen, Heerhugowaard (NL)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/462,158

(22) Filed: Aug. 3, 2006

(51) Int. Cl. A61K 31/4745; A61K 9/14; A61K 31/205; A61K 31/137

(52) U.S. Cl. 424/46; 514/291; 514/554; 514/649

(57) ABSTRACT

The present invention relates to the use of tiotropium salts in combination with salts of salmeterol for the manufacture of a medicament for the treatment of dyspnea.

### RELATED APPLICATION

This application claims priority to European Patent Application No. 05 107 267.6, filed Aug. 6, 2006, the content of which is incorporated by reference in its entirety.

### FIELD OF THE INVENTION

The present invention relates to the use of tiotropium salts in combination with salts of salmeterol for the manufacture of a medicament for the treatment of dyspnea.

### DESCRIPTION OF THE INVENTION

Medicament combinations based on anticholinergics and beta-2-agonists are known in the art. The combination of long acting anticholinergics with long acting beta-2-agonists is disclosed in WO00/69468. The specific combination of tiotropium salts with salts of salmeterol is also known from WO 02/38154 and WO 04/058233.

These protective rights also disclose pharmaceutical compositions comprising salts of tiotropium and salts of salmeterol.

Surprisingly, it has been found that the combinations of tiotropium salts with salts of salmeterol are particularly useful in the preparation of a medicament for the treatment of dyspnea.

Dyspnea is a term known in the art. The term dyspnea is used to characterize a subjective experience of breathing discomfort that consists of qualitatively distinct sensations that vary in intensity. The experience derives from interactions among multiple physiological, psychological, social and environmental factors, and may induce secondary physiological and behavioural responses (see hereto: American Thoracic Society, Dyspnea: mechanisms, assessment, and management. A consensus statement, Am J Respir Crit Care Med 159: 321-340, 1999).

Therefore, the invention relates to the use of a tiotropium salt 1 together with a salmeterol salt 2 for the manufacture of a medicament for the treatment of dyspnea.

In another embodiment the invention relates to the use of a tiotropium salt 1 for the manufacture of a salmeterol salt 2 containing medicament for the treatment of dyspnea.

In a yet another preferred embodiment the invention relates use of a salmeterol salt 2 for the manufacture of a tiotropium salt 1 containing medicament for the treatment of dyspnea.

In another embodiment the invention relates to a method for the treatment of dyspnea, comprising the administration of a therapeutically effective amount of a tiotropium salt 1 together with a therapeutically effective amount of a salmeterol salt 2 to a patient in need thereof. The term “therapeutically effective amount” shall mean that amount of a drug or pharmaceutical agent that will elicit the biological or medical response of a tissue, system, animal or human that is being sought by a researcher or clinician.

The combined administration of a 1 and 2 may occur via a single active substance formulation but also via the successive administration of the two active substances in separate formulations. It is preferred according to the invention to administer the two active substance ingredients simultaneously in a single formulation.

The name tiotropium is intended to refer to the free cation (1′) for the purposes of the present invention. The tiotropium salts 1 which may be used within the scope of the present invention include the compounds which contain, in addition to the cation tiotropium 1′ an anion with a single negative charge, preferably an anion selected from among chloride, bromide, iodide, methanesulphonate and para-toluenesulphonate. Within the scope of the present invention, of all the tiotropium salts, the methanesulphonate, chloride, bromide or iodide are preferred, the methanesulphonate or bromide being of particular importance. Tiotropium bromide is of exceptional importance according to the invention.

By salts of salmeterol 2 are meant, according to the invention, pharmaceutically acceptable acid addition salts selected from among the salts of hydrochloric acid, hydrobromic acid, sulphuric acid, phosphoric acid, methanesulphonic acid, acetic acid, fumaric acid, succinic acid, lactic acid, citric acid, tartaric acid, xinafonic acid or maleic acid. According to the invention, the salmeterol salts 2 selected from among hydrochloride, hydrobromide, sulphate, phosphate and xinafoate are preferred. The salmeterol xinafoate salt 2 is particularly preferred.

In the combinations of active substances according to the invention consisting of 1 and 2 the ingredients 1 and 2 may be in the form of their enantiomers, mixtures of enantiomers or in the form of the racemates. The active substances 1 and 2 may optionally be in the form of the solvates or hydrates thereof.

Suitable inhalable preparations for the administration of 1 and 2 include inhalable powders, metering aerosols containing propellant gases or inhalable solutions free from propellant gases. Inhalable powders according to the invention containing the active substance combination of 1 and 2 may consist solely of the abovementioned active substances or of a mixture of the abovementioned active substances with physiologically acceptable adjuvants. The preparations may contain the active substance combination of 1 and 2 either together in one preparation or in two separate preparations.

The international patent applications WO00/69468, WO 02/38154 and WO 04/058233 which are hereby incorporated by reference in their entirety, disclose preferred pharmaceutical formulations and compositions that are also suitable for the use according to the invention.

The proportions in which the two active substances 1 and 2 may be used in the combinations of active substances according to the invention are variable. The active substances 1 and 2 may optionally be in the form of the solvates or hydrates thereof. Depending on the choice of the salts 1 or 2 the weight ratios which may be used for the purposes of the present invention vary on account of the different molecular weights of the various salt forms. Consequently, the weight ratios specified hereinafter are based on the tiotropium cation 1′ and the free base of salmeterol 2′. The combinations of active substances according to the invention may contain 1′ and 2′ in weight ratios in the range from 1:300 to 30:1, preferably from 1:230 to 20:1, particularly preferably from 1:150 to 10:1, more preferably from 1:50 to 5:1, particularly preferably from 1:35 to 2:1. Of particular interest according to the invention are pharmaceutical compositions containing the combination of 1′ and 2′ in a weight ratio in the range from 1:25 to 1:1, preferably in the range from 1:10 to 1:2, particularly preferably in the range from 1:5 to 1:2.5.

For example, without restricting the scope of the invention, preferred combinations of 1 and 2 according to the invention may contain tiotropium 1′ and salmeterol 2′ in the following weight ratios: 1:40; 1:20; 1:1.1; 1:10; 1:5.6; 1:5; 1:2.8; 1:2.5; 1:1.4; 1:1.25; 1.44:1, 1.6:1.

The pharmaceutical compositions according to the invention containing the combinations of 1 and 2 are normally used so that tiotropium 1′ and salmeterol 2′ are administered together in doses of 0.01 to 10000 μg, preferably 0.1 to 2000 μg, particularly preferably from 1 to 1000 μg, more preferably from 5 to 500 μg, preferably, according to the invention, from 10 to 200 μg, preferably from 20 to 100 μg, most preferably from 30 to 70 μg per single dose.

For example, combinations of 1 and 2 according to the invention contain an amount of tiotropium 1′ and salmeterol 2′ such that the total dosage per single dose is 30 μg, 35 μg, 45 μg, 55 μg, 60 μg, 65 μg, 90 μg, 105 μg, 110 μg, 110 μg, 140 μg or similar. In these dosage ranges the active substances 1′ and 2′ are present in the weight rations described hereinbefore.

For example, without restricting the scope of the invention, the combinations of 1 and 2 according to the invention may contain an amount of tiotropium 1′ and salmeterol 2′ such that 5 μg of 1′ and 25 μg of 2′, 5 μg of 1′ and 50 μg of 2′, 5 μg of 1′ and 100 μg of 2′, 5 μg of 1′ and 200 μg of 2′, 10 μg of 1′ and 25 μg of 2′, 10 μg of 1′ and 50 μg of 2′, 10 μg of 1′ and 100 μg of 2′, 100 μg of 1′ and 200 μg of 2′, 18 μg of 1′ and 25 μg of 2′, 18 μg of 1′ and 50 μg of 2′, 18 μg of 1′ and 100 μg of 2′, 18 μg of 1′ and 200 μg of 2′, 20 μg of 1′ and 25 μg of 2′, 20 μg of 1′ and 50 μg of 2′, 20 μg of 1′ and 100 μg of 2′, 20 μg of 1′ and 200 μg of 2′, 36 μg of 1′ and 25 μg of 2′, 36 μg of 1′ and 50 μg of 2′, 36 μg of 1′ and 100 μg of 2′, 36 μg of 1′ and 200 μg of 2′, 40 μg of 1′ and 25 μg of 2′, 40 μg of 1′ and 50 μg of 2′, 40 μg of 1′ and 100 μg of 2′ or 40 μg of 1′ and 200 μg of 2′ are administered per single dose.

The combinations of active substances 1 and 2 according to the invention are preferably administered by inhalation. For this purpose, the ingredients 1 and 2 have to be incorporated in inhalable preparations.

Suitable inhalable preparations include inhalable powders, metering aerosols containing propellant gases or inhalable solutions free from propellant gases. Inhalable powders according to the invention containing the active substance combination of 1 and 2 may consist solely of the abovementioned active substances or of a mixture of the abovementioned active substances with physiologically acceptable adjuvants. Within the scope of the present invention the term propellant-free solutions for inhalation also includes concentrates or sterile, ready-to-use solutions for inhalation. The preparations according to the invention may contain the active substance combination of 1 and 2 either together in one preparation or in two separate preparations. These preparations which may be used within the scope of the present invention are described in detail in the following section of the specification.

A) Inhalable powders containing the active substance combinations of 1 and 2 according to the invention:

The powders for inhalation according to the invention may contain 1 and 2 either on their own or in admixture with suitable physiologically harmless adjuvants.

If the active substances 1 and 2 are present in admixture with physiologically harmless adjuvants, the following physiologically harmless adjuvants may be used to prepare these inhalable powders according to the invention: monosaccharides (e.g. glucose or arabinose), disaccharides (e.g. lactose, saccharose, maltose), oligo- and polysaccharides (e.g. dextranes), polyalcohols (e.g. sorbitol, mannitol, xylitol), salts (e.g. sodium chloride, calcium carbonate) or mixtures of these adjuvants with one another. Mono- or disaccharides are preferably used, the use of lactose or glucose, particularly but not exclusively in the form of their hydrates, being preferred. The particularly preferred adjuvant according to the invention is lactose, most preferably lactose monohydrate.

Within the scope of the powders for inhalation according to the invention the adjuvants have a maximum mean particle size of up to 250 μm, preferably between 10 and 150 μm, particularly preferably between 15 and 80 μm. If desired it may be useful to add finer adjuvant fractions having a mean particle size of 1 to 9 μm to the abovementioned adjuvants. These latter finer adjuvants are also selected from the abovementioned group of adjuvants which may be used. Finally, in order to prepare the powders for inhalation according to the invention, micronised active substance 1 and 2, preferably having an average particle size of 0.5 to 10 μm, particularly preferably from 1 to 6 μm, is added to the adjuvant mixture. Processes for preparing the powders for inhalation according to the invention by grinding and micronising and finally mixing the ingredients together are known from the prior art. The powders for inhalation according to the invention may be prepared and administered either in the form of a single powder mixture which contains both 1 and 2, or in the form of separate inhalable powders which contain only 1 and 2.

The inhalable powders according to the invention can be administered using inhalers known from the prior art.

Inhalable powders according to the invention which contain a physiologically harmless adjuvant in addition to 1 and 2 may for example be administered using inhalers which meter a single dose from a reservoir by means of a measuring chamber, as described in U.S. Pat. No. 4,570,630A, or by other devices as described in DE 36 25 685 A. Preferably, the inhalable powders according to the invention which contain physiologically harmless adjuvant in addition to 1 and 2 are packed into capsules (to form so-called inhalettes), which are used in inhalers such as those described, for example, in WO 94/28958. If the inhalable powders according to the invention are to be packed into capsules (inhalettes) as in the preferred application mentioned above, fillings of 1 to 30 mg, preferably from 3 to 20 mg, preferably 5 to 10 mg of inhalable powder per capsule are suggested. According to the invention, these contain the dosages specified above for 1′ and 2′ either together or separately per single dose.

B) Inhalable aerosols containing propellant, comprising the active substance combinations of 1 and 2 according to the invention:

Inhalable aerosols containing propellant according to the invention may contain 1 and 2 dissolved in the propellent gas or in dispersed form. 1 and 2 may be present in separate preparations or in a combined preparation, with 1 and 2 either both dissolved, both dispersed or only one component dissolved while the other is present in dispersed form. The propellent gases which can be used to prepare the inhalable aerosols according to the invention are known from the prior art. Suitable propellent gases are selected from among hydrocarbons such as n-propane, n-butane or isobutane and halohydrocarbons such as chlorinated and/or fluorinated derivatives of methane, ethane, propane, butane, cyclopropane or cyclobutane. The abovementioned propellent gases may be used on their own or in mixtures thereof. Particularly preferred propellent gases are halogenated alkane derivatives selected from among TG11, TG12, TG134a and TG227. Of the abovementioned halogenated hydrocarbons, TG134a (1,1,1,2-tetrafluoroethane) and TG227 (1,1,1,2,3,3,3-heptafluoropropane) and mixtures thereof are preferred according to the invention.

The propellant-gas-containing inhalable aerosols according to the invention may also contain other ingredients such as cosolvents, stabilisers, surface-active agents (surfactants), antioxidants, lubricants and means for adjusting the pH. All these ingredients are known in the art.

The propellant-gas-containing inhalable aerosols according to the invention may contain up to 5% by weight of active substance 1 and/or 2. Aerosols according to the invention contain, for example, 0.002 to 5% by weight, 0.01 to 3% by weight, 0.015 to 2% by weight, 0.1 to 2% by weight, 0.5 to 2% by weight or 0.5 to 1% by weight of active substance 1 and/or 2.

If the active substances 1 and/or 2 are present in dispersed form the particles of active substance preferably have a mean particle size of up to 10 μm, preferably from 0.1 to 5 μm, particularly preferably from 1 to 5 μm.

The abovementioned propellant-gas-containing inhalable aerosols according to the invention can be administered by means of inhalers known in the art (MDIs=metered dose inhalers). Accordingly, a further aspect of the present invention relates to pharmaceutical compositions in the form of propellant-gas-containing aerosols as described above combined with one or more inhalers suitable for administering these aerosols. Furthermore, the present invention relates to inhalers, characterised in that they contain the propellant-gas-containing aerosols according to the invention as described above.

The present invention also relates to cartridges which are fitted with a suitable valve and can be used in a suitable inhaler and which contain one of the abovementioned propellant-gas-containing inhalable aerosols according to the invention. Suitable cartridges and methods of filling these cartridges with the propellant-gas-containing inhalable aerosols according to the invention are known from the prior art.

C) Propellant-free inhalable solutions containing the active substance combinations of 1 and 2 according to the invention:

It is particularly preferable for the active substance combination according to the invention to be administered in the form of propellant-free solutions for inhalation. Suitable solvents for this include aqueous or alcoholic, preferably ethanolic solutions. The solvent may be water on its own or a mixture of water and ethanol. The relative proportion of ethanol to water is not restricted, but the maximum limit is preferably up to 70 percent by volume, particularly up to 60 percent by volume and most preferably up to 30 percent by volume. The remaining percent by volume are made up with water. The preferred solvent is water without the addition of ethanol. The solutions containing 1 and 2, separately or together, are adjusted to a pH of 2 to 7, preferably 2 to 5, particularly preferably 2.5 to 3.5, with suitable acids. Most preferably, inhalable solutions according to the invention which contain 1 and 2 together have a pH of about 2.9. This pH may be achieved using acids selected from among inorganic or organic acids. Examples of particularly suitable inorganic acids include hydrochloric acid, hydrobromic acid, nitric acid, sulphuric acid and/or phosphoric acid. Examples of particularly suitable organic acids include: ascorbic acid, citric acid, malic acid, tartaric acid, maleic acid, succinic acid, fumaric acid, acetic acid, formic acid and/or propionic acid and others. Preferred inorganic acids are hydrochloric acid and sulphuric acid. It is also possible to use the acids which are forming an acid addition salt with the active substance or, in the case of combined preparations, with one of the active substances. Of the organic acids, ascorbic acid, fumaric acid and citric acid are preferred. If desired, mixtures of the abovementioned acids may also be used, particularly in the case of acids which have properties other than their acidifying properties, e.g. as flavourings, antioxidants or complexing agents, such as citric acid or ascorbic acid, for example. According to the invention, it is particularly preferred to use hydrochloric acid to adjust the pH.

According to the invention, there is no need to add editic acid (EDTA) or one of the known salts thereof, sodium edetate, to the present formulation as a stabiliser or complexing agent.

Other embodiments contains these compound(s).

In a preferred embodiment of this kind, the content based on sodium edetate is less than 100 mg/100 ml, preferably less than 50 mg/100 ml, most preferably less than 20 mg/100 ml.

Inhalable solutions in which the content of sodium edetate is 0 to 10 mg/100 ml are generally preferred.

Co-solvents and/or other adjuvants may be added to the propellant-free inhalable solutions according to the invention.

Preferred co-solvents are those which contain hydroxyl groups or other polar groups, e.g. alcohols—particularly isopropylalcohol, glycols—particularly propyleneglycol, polyethyleneglycol, polypropyleneglycol, glycolether, glycerol, polyoxyethylene alcohols and polyoxyethylene fatty acid esters.

By excipients and additives is meant, in this context, any pharmacologically acceptable substance which is not an active substance, but can be formulated together with the active substance(s) in the pharmacologically suitable solvent, in order to improve the qualitative properties of the active substance formulation. Preferably, these substances do not have any appreciable pharmacological effects or at least have no undesirable effects in the context of the intended therapy. The excipients and additives include e.g. surfactants such as e.g. soya lecithin, oleic acid, sorbitan esters such as polysorbates, polyvinylpyrrolidone, other stabilisers, complexing agents, antioxidants and/or preservatives which guarantee or extend the shelf life of the finished pharmaceutical formulation, flavourings, vitamins and/or other additives known in the art. The additives also include pharmacologically harmless salts such as sodium chloride, for example, as isotonic agents.

The preferred adjuvants include antioxidants, such as ascorbic acid, for example, unless it has already been used to adjust the pH, vitamin A, vitamin E, tocopherols and similar vitamins or provitamins occurring in the human body.

Preservatives can be used to protect the formulation from contamination with pathogens. Suitable preservatives are those known from the prior art, particularly cetylpyridinium chloride, benzalkonium chloride or benzoic acid or benzoates such as sodium benzoate in the concentration known from the prior art. The abovementioned preservatives are preferably present in concentrations of up to 50 mg/100 ml, particularly between 5 and 20 mg/100 ml.

Preferred formulations contain only benzalkonium chloride and sodium edetate in addition to the solvent water and the active substance combination of 1 and 2. In another preferred embodiment, sodium edetate is omitted.

The propellant-free inhalable solutions according to the invention may be administered particularly using inhalers which are able to nebulise a small amount of a liquid formulation in the therapeutically necessary dose within a few seconds to form an aerosol suitable for therapeutic inhalation. Within the scope of the present invention, nebulisers are preferred in which a quantity of less than 100 μL, preferably less than 50 μL, particularly preferably between 20 and 30 μL of active substance solution can be nebulised, preferably in one operation, to produce an aerosol having an average particle size of less than 20 μm, preferably less than 10 μm, in such a way that the inhalable part of the aerosol corresponds to the therapeutically effective amount.

A device of this kind for the propellant-free administration of a metered amount of a liquid pharmaceutical composition for inhalation is described in detail, for example, in International Patent Application WO 91/14468 and also in WO 97/12687 (particularly FIGS. 6a and 6b). The nebulisers (devices) described therein are also known by the name Respimat®.

I claim:

1. A method of treating dyspnea comprising administering to a patient in need thereof a therapeutically effective amount of a tiotropium salt 1 together with a therapeutically effective amount of a salmeterol salt 2.

2. The method according to claim 1, wherein the tiotropium salt 1 is selected from the group consisting of chloride, bromide, iodide, methanesulphonate and para-toluenesulphonate.

3. The method according to claim 1 or 2, wherein the salmeterol salt 2 is selected from the group consisting of hydrochloric acid, hydrobromic acid, sulphuric acid, phosphoric acid, methanesulphonic acid, acetic acid, fumaric acid, succinic acid, lactic acid, citric acid, tartaric acid, xinafonic acid and maleic acid.
